(19) United States
(12) Patent Application Publication
(10) Pub. No.: US 2025/0195347 A1
(43) Pub. Date: Jun. 19, 2025

(54) PEROXIDE HYDROGEL COMPOSITIONS AND THEIR USE FOR FIBER BLEACHING

(71) Applicant: HENKEL AG & CO. KGAA, Düsseldorf (DE)

(72) Inventors: Ranju Prasad MANDAL, Düsseldorf (DE); Daniela KESSLER-BECKER, Düsseldorf (DE); Jennifer S. HAGHPANAH, New York, NY (US); Jeffrey S. BANKERS, Stamford, CT (US)

(21) Appl. No.: 18/847,255

(22) PCT Filed: Mar. 18, 2023
(86) PCT No.: PCT/US2023/015585

§ 371 Date: Sep. 15, 2024

Related U.S. Application Data
(60) Provisional application No. 63/269,576, filed on Mar. 18, 2022.

(51) Int. Cl. A61K 8/22; A61K 8/04; A61K 8/24; A61K 8/25; A61Q 5/08

(52) U.S. Cl. CPC A61K 8/22; A61K 8/042; A61K 8/24; A61K 8/25; A61Q 5/08; A61K 2800/882

(57) ABSTRACT

Keratinous fiber lightening peroxide hydrogel compositions and a method for their preparation are provided. The hydrogel compositions include hydrogen peroxide, a metal silicate, and metal cation and have a pH of about 3 to about 4.5. A bleaching system includes the peroxide hydrogel compositions and an oxidative agent booster. Methods of use of the peroxide gel alone or in a system with a booster to lighten and/or bleach keratinous fibers, such as hair, result in less damage to the hair as compared to products in which the hydrogen peroxide is not contained in a hydrogel.

### CROSS-REFERENCE TO RELATED APPLICATIONS

The present application claims the benefit of U.S. Provisional Application No. 63/269,576 filed on Mar. 18, 2022 and entitled “PEROXIDE HYDROGEL COMPOSITIONS AND THEIR USE FOR FIBER BLEACHING”. The entire disclosure of this provisional application is incorporated herein by reference.

### FIELD OF THE INVENTION

The invention relates to peroxide hydrogel compositions for lightening keratin-containing fibers, including mammal keratin-containing-containing fibers, and more particularly human hair, and method for preparation of such compositions. Specifically, the invention concerns systems and methods of lightening and/or bleaching hair that include peroxide hydrogel compositions that can be combined with additional oxidizing agents and applied to hair.

### BACKGROUND OF THE INVENTION

Hair bleaching is a well-known process in the hair cosmetic field. Traditional hair bleaching systems involve the application of an oxidizing agent to the hair for a period of time to achieve a desired lighter hair shade. The oxidizing agent typically is a hydrogen peroxide solution in concentrations ranging from 6 to 12% by weight.

In these traditional bleaching systems, the hydrogen peroxide is applied to the hair under alkaline pH conditions, often created by inclusion of ammonia. This gradually lightens the shade of the hair by oxidizing the melanin that gives it color. To enhance the lightening efficacy of the hydrogen peroxide oxidizer, many traditional hair bleaching systems incorporate a persulfate salt as a “booster.”

However, oftentimes with the traditional alkaline bleaching systems, consumers experience undesired hair damage including poor hair feel and look, reduced hair strength, increased incidents of split ends and breakage. This is because with traditional bleaching systems, the hydrogen peroxide solution directly attacks the hair's melanin.

High alkaline pH is necessary for the deprotonation of hydrogen peroxide and the formation of the hydroperoxide anion (OOH) which is an important oxidant in the oxidative hair bleaching process. The hydroperoxide anion carries out nucleophilic attacks on melanin granules in order to lighten hair color, however this causes undesired hair damage such as poor hair feel and look, reduced hair strength, increased incidents of split ends and breakage.

The hydrogen peroxide used in a traditional hair bleaching system oxidatively cleaves the disulfide bonds found in human hair and converts cysteine residues to cysteic acid residues. Increased cysteic acid is considered to be a major cause of hair damage, and local changes in mechanical properties through cysteic acid formation have been presumed to affect the hair texture in an undesirable manner. Thus, the content of cysteic acid residue is a major indicator of hair damage after bleaching treatments.

It is an object of the present invention to eliminate the level of resulting hair damage associated with traditional hair bleaching systems.

Studies, for example, Ind. Eng. Chem. Res. 2017, 56, 9, 2607-2614, indicate that encapsulating hydrogen peroxide in silica hydrogels can tailor the release of hydrogen peroxide for an intended application. However, hydrogen peroxide encapsulation has yet to be used in hair lightening systems.

Thus, it is an object of the present invention to provide a peroxide hydrogel composition and its use in a system suitable for hair and other keratinous fibers, which lightens and/or bleaches fibers just as effectively as existing bleaching systems, but with less resulting damage.

### SUMMARY OF THE INVENTION

The foregoing objectives are achieved by fiber lightening peroxide hydrogel compositions, a bleaching system containing the peroxide hydrogel compositions and an oxidative agent booster, and methods of use of the gel and/or gel systems to lighten and/or bleach keratinous fibers, such as hair.

In one embodiment, a peroxide hydrogel composition suitable for keratinous fibers comprises about 5 to about 17% by weight hydrogen peroxide; a metal silicate; and a metal cation or a salt thereof. The composition has a pH of about 3 to about 6.5. In certain embodiments, the metal silicate is sodium silicate and may comprise about 15% to about 30% by weight of the composition. The metal cation is preferably a divalent cation salt present at about 0.1 to about 1% by weight, such as calcium chloride. The compositions can further comprise an organic acid, such as about 0.5 to about 2.5% by weight phosphoric acid.

In some embodiments, the peroxide hydrogel composition is used in a system with an oxidizing agent booster. When combined in a 1:1 to 1:2 ratio, the system has a pH of about 9 to 12 when measured at ambient conditions at about 25 degrees Celsius. The oxidizing agent booster can comprise a persulfate selected from inorganic salts of a peroxosulfuric acid and mixtures thereof, and typically will comprise about 5 to about 85% by weight of the persulfate. In some embodiments, the booster is a powder.

The peroxide composition and oxidizing agent booster can be provided as separate components in a kit, where the components are in a weight ratio of about 0.8:1 to about 1:2.5, more preferably 1:1 to about 1:2. The kit can further comprise at least one of an application aid, disposable gloves, and instructions for use.

Also provided is a process for producing a cosmetic peroxide containing hydrogel that is suitable for lightening hair comprising the steps of:
preparing an aqueous solution containing about 0.5 to about 1.5% by weight of a metal cation salt;
adding a metal silicate to the metal cation salt solution at a weight fraction of about 12.5 to about 25%;
adding a solution of a concentrated inorganic acid selected from phosphoric acid, citric acid, and ascorbic acid to the metal cation salt and metal silicate solution to obtain a sol;
adding about 5 to about 17% by weight hydrogen peroxide to the sol;
exchanging the metal silicate cations in the peroxide-containing sol with hydrogen ions
adjusting the pH of the ion-exchanged sol to about 3 to 6.5 to form a hydrogel. The process does not include a step of heating the sol to form the hydrogel.

Advantageously, the method of preparing the peroxide hydrogel does not require drying of the gel, either by extraction, evaporation or heating.

Typically, the step of exchanging is performed with an ion exchange resin, and the method may include the step of separating the ion exchange resin from the ion-exchanged sol. The ion exchange resin is added at molar ratio of metal in the metal cation salt to metal in the metal silicate of about 0.5-1.5:5-15, most preferably 1:10.

The metal cation is preferably calcium chloride; the acid is preferably phosphoric acid.

The invention also includes a method for lightening keratinous fibers comprising applying the peroxide hydrogels described herein to a keratinous fiber for 5 to 60 minutes. A method of bleaching comprises first mixing the peroxide hydrogel with an oxidizing agent booster to form a ready-to-use mixture and then applying the ready-to-use mixture to the keratinous fiber. The peroxide gel and oxidizing agent are typically combined in a 1:1 to 1:2 ratio.

The methods may comprise applying heat and/or foils to the hair for at least part of the application time. The hair is typically rinsed with water, and/or a cleaning agent and/or a conditioning agent.

When the fiber is hair, the methods advantageously decrease damage to the lightened or bleached hair as compared to a method in which the hydrogen peroxide is not contained in a hydrogel composition.

### BRIEF DESCRIPTION OF THE DRAWINGS

The foregoing summary, as well as the following detailed description of preferred embodiments of the invention, will be better understood when read in conjunction with the appended drawings. For the purpose of illustrating the invention, there is shown in the drawings embodiments which are presently preferred. It should be understood, however, that the invention is not limited to the precise arrangements and instrumentalities shown. In the drawings:

FIGS. 1A and 1B are a photograph showing ΔE (DE) and ΔL (DL) values of hair bleached with a system of peroxide hydrogel composition and booster (1A) versus a traditional non-hydrogel bleaching system (1B) as described in Examples 2 and 4 after 60 minutes.

FIGS. 2A-2C are SEM images at various magnifications showing the surface of virgin hair bleached with a traditional bleaching system.

FIGS. 2D-2F are SEM images at various magnifications showing the surface of virgin hair bleached with a system containing a peroxide hydrogel composition and an oxidative agent booster combined in a 1:2 ratio.

FIGS. 3A-3C are SEM images showing the surface of untreated virgin hair, virgin hair lightened using a traditional peroxide developer (no booster), and virgin hair bleached with a traditional developer plus a booster, respectively, after application for 60 minutes.

FIGS. 3D-3F are SEM images of portions of the hair samples of FIGS. 3A-3C, respectively, at a higher magnification.

FIGS. 4A and 4D are SEM images showing the surface of hair lightened using a peroxide hydrogel composition (no booster), after 60 minutes of application as described in Example 2.

FIGS. 4B and 4C are SEM images at different magnifications showing the surface of hair bleached using a system of peroxide hydrogel composition and a booster in a 1:1 ratio, after 60 minutes of application as described in Example 4.

FIGS. 4E and 4F are SEM images at different magnifications showing the surface of hair bleached using a system of peroxide hydrogel composition and a booster in a 1:2 ratio, after 60 minutes of application as described in Example 4.

FIG. 5A shows two microscopic images of hair bleached using a peroxide hydrogel bleaching composition with a booster, after 60 minutes of application.

FIG. 5B shows two microscopic images of hair bleached using a traditional bleaching system with a booster, after 60 minutes of application.

### DETAILED DESCRIPTION OF THE INVENTION

The following detailed description is merely exemplary in nature and is not intended to limit the compositions and systems contemplated herein, the methods for forming the hydrogel or the applicant and uses of the composition and systems. Furthermore, there is no intention to be bound by any theory presenting in the preceding background or following detailed description.

The present disclosure provides compositions and methods for lightening and bleaching hair or other keratin-containing fibers, in particular human hair of the head. The compositions and methods are unique because, in addition to lightening or bleaching the keratin fiber, they dramatically improve the quality and durability of lightened/bleached hair and minimize hair damage. More specifically, the hydrogel bleaching system disclosed herein is capable of bleaching hair while minimizing the amount and types of hair damage associated with traditional bleaching systems, i.e., poor hair feel and look, reduced hair strength, increased incidents of split ends and breakage.

Peroxide containing hydrogel compositions, described in further detail herein, can be used to selectively lighten and/or bleach keratinous fiber without negatively impacting the tensile strength of the fiber and/or increasing cysteic acid formation. A hair bleaching system as contemplated herein is comprised of a peroxide containing hydrogel composition (preferably, a silica-based hydrogel that encapsulates peroxide within the hydrogel), and a separate oxidative agent (i.e., “booster”). Such systems advantageously bleach a keratinous fiber without negatively impacting the tensile strength and/or increasing cysteic acid formation.

The term “lightening” used herein in association with keratinous fibers encompasses any form of color change of the fibers in which the keratin fibers have a lighter color compared to the color present before the agent was used.

The lighter coloring of the hair is brought about by the oxidizing agent or agents present. To achieve moderate lightening effects, hydrogen peroxide is the oxidizing agent of choice. To achieve “bleaching,” hydrogen peroxide is used together with stronger oxidizing agents such as persulfates (sodium persulfate, potassium persulfate, ammonium persulfate).

As contemplated herein, keratin or keratinous fibers are defined as furs, wool, feathers, and especially human hair. Although the agents as contemplated herein are primarily suitable for bleaching and/or lightening fibers containing keratin, there is nothing in principle to prevent their use in other areas.

In the systems herein for lightening keratin-containing fibers, the terms “lift” or “lifting” relates to the lightening of the hair. Use of “lift” and “lightening” herein mean the same thing and the terms may be used interchangeably.

As used herein, the term “peroxide containing” means containing a peroxide oxidizing agent. The preferred oxidizing agent is hydrogen peroxide, however, other organic and inorganic peroxides, including salts and hydrates thereof are encompassed. In the present system, however, hydrogen peroxide is most preferred.

Encapsulating hydrogen peroxide (H₂O₂) in silica or other hydrogels has yet to be applied in keratinous fiber lightening systems or as a hair bleaching method. Encapsulating hydrogen peroxide in silica hydrogels is an environmentally friendly and cost-effective method. Surprisingly, it has been found that embedding or suspending hydrogen peroxide within a hydrogel composition lightens hair just as effectively as traditional lightening/bleaching products and systems with less resulting hair damage.

As used herein “hydrogel” refers to a crosslinked hydrophilic polymer that does not dissolve in water. Hydrogels as contemplated herein are typically formulated using sol-gel chemistry. Sol-gel is a wet-chemical process that involves the formation of an inorganic colloidal suspension (i.e., “sol”) and gelation of the sol in a continuous liquid phase (i.e., “gel”) to form a three-dimensional network structure. The sol is formed using an inorganic or organometallic gel precursor.

Inorganic gel precursors suitable for use herein include silicates, preferably in salt form. The term is not meant to be limiting. A preferred silicate for use herein is sodium silicate, and sodium silicate may be referred to generally herein for exemplary purposes, but it should be understood that other silicates that demonstrate the same or similar properties may be used within the scope of the invention. Examples of such silicates that may be used as the in the present invention include aluminum silicate, calcium silicate, magnesium aluminum silicate, magnesium silicate, magnesium trisilicate, sodium magnesium silicate, zirconium silicate, attapulgite, bentonite, Fuller's earth, hectorite, kaolin, lithium magnesium silicate, lithium magnesium sodium silicate, montmorillonite, pyrophyllite and zeolite, the latter of which are clay-like ingredients that are based on silicates.

The precursors used in sol-gel processes can also be organometallic, such as the tetramethyl orthosilicate and tetraethyl orthosilicate compounds (of formula Si(OCH₃)₄ and Si(OC₂H₅)₄, respectively, generally referred to as TMOS and TEOS.

Sodium silicate is the most commonly used aqueous silicate in the sol-gel chemistry. Silanol (SiOH) groups are formed by removing Na+ ions from sodium silicate with an ion-exchange resin. In preferred embodiments, the hydrogel of the present invention is formed using sodium silicate precursor and replacing the sodium ions with hydrogen using an ion-exchange resin.

The amount of gel precursor will typically range from about 15 to about 30% by weight of the hydrogel composition.

The structure of hydrogels can be manipulated using different types and amounts of crosslinking agents, such as metal cations. The use of different types and/or amounts of cross-linkers impacts the gel time and activation energy for gel formation, and tailors the release of encapsulated H₂O₂ for an intended application. In addition, the satiability of H₂O₂ within a hydrogel can be manipulated by modifying the metal ions present in the sol and/or by modifying the pH of the sol.

Metal cations that are suitable for manipulating cross-linking of the gelling agents and release of peroxide as contemplated herein include monovalent, divalent, and trivalent metals and salts thereof.

Examples of monovalent cations include sodium (Na⁺), potassium (K⁺), and lithium (Li⁺).

Examples of divalent cations include magnesium (Mg2⁺), strontium (Sr²⁺), and calcium (Ca²⁺).

Examples of trivalent cations include Aluminum (Al³⁺), lanthanum (La³⁺), and yttrium (Y³⁺).

Suitable salts of metal ions include chlorides, nitrate. Preferably, a water-soluble salt is utilized.

Experiments performed by the inventors showed that trivalent metal ions may reduce the amount of color change imparted to hair by hydrogen peroxide and/or affect the stability of the hydrogel. Moreover, it is expected that monovalent cations may not produce gels with long term stability. Thus, in preferred embodiments, the metal ions present in the hydrogel of the present invention are divalent metal ions, specifically, Ca²⁺. In certain embodiments the metal cation is provided by calcium chloride.

In other embodiments, where a slower release is desired, the hydrogel of the present invention is comprised of Mg²⁺ metal ions.

The amount of metal cation will typically range from about 0.1 to about 1.5% by weight of the hydrogel composition, preferably about 0.2 to about 1.0% by weight, most preferably about 0.25% to about 0.5% by weight of the hydrogel composition. In particularly preferred embodiments, the hydrogel composition comprises about 0.6 to about 0.8% by weight calcium chloride.

The pH level has been found to impact both hydrogel formation and release of encapsulated H₂O₂ within the hydrogel. Silicate salts are water soluble but only at high pH levels such as 10.9 to 12. At lower pH, such salts are neutralized and known to precipitate out of solution as a silicic salt. Nonetheless, the peroxide hydrogel composition described herein preferably has an acidic pH value, in particular a pH value in the range of from about 3 to about 7, measured at about 25° C. More preferably, the hydrogel composition has a pH of about 3 to 6.5, most preferably about 3.5 to 6.5.

Suitable pH adjusting agents include organic acids, preferably the organic acids is selected from phosphoric acid, citric acid, and ascorbic acid.

In certain embodiments, the hydrogel of the present invention is formed at a pH of around 4 after removal of resins, e.g., through decanting, and the addition of a low pH acid, preferably, orthophosphoric acid.

In some embodiments, the hydrogels as discussed herein comprise alternative pH-raising ingredients including sodium carbonate and sodium hydroxide.

In certain preferred embodiments, the hydrogel will comprise about 0.5 to about 2.5% by weight phosphoric acid, preferably added over two or more steps.

The hydrogels will typically comprise about 65 to about 80% by weight water.

The hydrogel composition may include other optional ingredients included in traditional hair lightening compositions.

For instance, the composition may further contain as an optional component at least one amino acid selected from arginine, lysine, histidine or at least one of the salts of these amino acids. Arginine is highly preferred. As contemplated herein, mixtures of arginine and lysine may be particularly preferred. Among the salts of arginine, lysine or histidine preferred by the present disclosure are the alkali metal salts and alkaline earth metal salts, in particular the salts of lithium, sodium, potassium, magnesium and calcium, as well as hydrohalides, in particular hydrochlorides, and mixtures of these salts. As contemplated herein, lysine hydrochloride is a particularly preferred amino acid salt.

As contemplated herein, particularly preferred amino acid mixtures are selected from arginine/lysine hydrochloride and arginine/lysine. The amino acids, selected from arginine, lysine, histidine, and their salts as contemplated herein, may also contain water of crystallization.

Other optional components may include cationic surfactants (such as amines), nonionic surfactants, such as fatty acids or alcohols, polyethylene glycols, anionic surfactants and similar materials which are known in the art for this purpose and suitable for use in, compatible with and stable within hair coloring compositions.

Further optional components include dispersants known or to be developed in the art, thickeners, rheological agents, chelating agents, antioxidants, conditioning agents stable within the composition and system, viscosity modifiers, emulsifiers, fragrance components, preservatives, and similar materials.

In some preferred embodiments, the hydrogel composition is comprised of, consists essentially of, or consists of sodium silicate, calcium chloride, hydrogen peroxide, orthophosphoric acid and water. In preparation of the hydrogel, most of the sodium ions are exchanged with hydrogen ions.

The hydrogels can be prepared using the sol-gel method.

In one embodiment, the peroxide hydrogel can be prepared by first preparing an aqueous solution containing about 0.5 to about 1.5% by weight of a metal cation salt. A metal silicate is added to the metal cation solution at a weight fraction of about 12.5 to about 25%. A solution of a concentrated inorganic acid selected from sulfuric, hydrochloric, nitric and phosphoric acid is added to the metal cation salt and metal silicate solution to obtain a sol. Hydrogen peroxide is added to the sol. Preferably, to be suitable for hair, the concentration of hydrogen peroxide is about 5 to about 17% by weight. Thereafter, an ion exchange resin, such as AMBERLITE IR 120 (Hydrogen form) is added to the sol. The amount of ion exchange resin will affect the ability to separate the gel from the resin. Preferably, a hydrogen ion exchange resin is used at about 4% w/w to 10% w/w, preferably about 4.5% w/w to about 9% w/w, most preferably about 4.5% w/w. The mixture is stirred for several minutes, and resins are removed from the gel. The pH is adjusted with an inorganic acid to about 3.0 to about 4.5, preferably about 3.5 to about 4.5, most preferably about 4, to obtain the final gel.

It is desirable to add the inorganic acid in two separate steps or it becomes difficult to separate the resins from the final gel. Moreover, the fraction of metal silicate is critical to obtain a suitable rigid and stable gel for application to keratinous fibers. The method is advantageous in that it does not require heating or evaporation of liquid to form the gel.

The peroxide hydrogel lightening composition may be used independently or in other systems and can be provided as part of a system for combination and application to hair. The hydrogel composition may be sold as an independent product or together as a system in the form of a kit including user instructions setting forth the steps for combining and applying the compositions and combined system to hair following the method as described below.

In a bleaching system, an oxidizing agent, such as persalts, especially persulfate salts, preferably potassium sulfate and/or ammonium persulfate, should be added to the peroxide hydrogel composition so that it will make up typically about 5 to about 85 weight percent, and preferably about 10 to about 70 percent by weight. In one embodiment a bleaching powder contained about 42 percent by weight of persulfates. Dark, dark brown or black hair can be lightened by from about 4 to about 6 shades in one step. The hydrogen peroxide and the persalts are stored separately until use in order not to deactivate the persalts prematurely. The hydrogel composition (i.e., “gel”) and oxidizing agent (i.e., persalts (“booster”)) are combined before application to keratinous fibers to form a ready-to-use mixture.

The ready-to-use mixtures are prepared immediately before use on hair by mixing two preparations (A) and (B) or by mixing three preparations (A) and (B) and (C). In the case of ready-to-use mixtures that are mixed from more than two preparations to form a finished application mixture, it can be irrelevant whether two preparations are first mixed with one another and then the third preparation is added and mixed in, or whether all preparations are brought together and mixed. Mixing can be done by stirring in a bowl or beaker or by shaking in a sealable container.

In preferred embodiments, the peroxide hydrogel composition of the present invention (A) is used in connection with a bleaching or oxidizing agent (B), which when formulated is typically referred to as a “booster.”

The oxidizing agent is chosen from peroxides, persulfates, perborates, percarbonates, or mixtures thereof.

In certain preferred embodiments, the oxidizing agent comprises persulfates chosen from inorganic salts of a peroxosulfuric acid and mixtures thereof, preferably sodium persulfate, potassium persulfate, ammonium persulfate, or mixtures thereof. Peroxosulfuric acids are peroxodisulfuric acid and peroxomonosulfuric acid (Caro's acid).

The oxidizing agent is typically present from about 5-85% by weight, preferably from about 10-70% by weight, based on the weight of the booster. In some embodiments the persulfates of the oxidizing agent are present in about 42% by weight.

In some preferred embodiments the booster is a powder and will commonly be called a “blonding powder.”

As contemplated herein, the term “powder” or “powdery” means a solid, free-flowing dosage form including individual particles, solid at about 25° C. and about 1013 mbar, in which the individual particles have particle sizes ranging from about 0.1 μm to a maximum of about 1.6 mm The particle size distribution can preferably be determined by laser diffraction measurement according to ISO 13320-1 (2009). If necessary, the particle size of the blonding powder can be adjusted by physical treatment, such as sieving, pressing, granulating or pelletizing, or by the addition of certain auxiliary substances, to meet the requirements of the blonding powder, e.g., to improve the miscibility of the individual powder components or the miscibility of the blonding powder with a hydrogen peroxide preparation.

Blonding powders preferred according to the present disclosure have a bulk density in the range of from about 400 to about 1000 g/l (grams/liter), preferably from about 450 to about 900 g/l, especially preferred from about 550 to about 820 g/l. The bulk density is preferably determined according to EN ISO 60 (version January 2000) or DIN ISO 697 (version January 1984).

As contemplated herein, the booster is preferably composed in such a way that in mixture with the aforementioned peroxide hydrogel composition (i.e., the ready-to-use bleaching mixture), results in an alkaline pH value, preferably a pH value of from about 8.5 to about 12, particularly preferably a pH value of from about 9.0 to about 11.5, extremely preferably a pH value of from about 9.5 to about 11.0, in each case measured at about 25° C. Alternatively, the booster may be applied to hair followed by subsequent application of the peroxide gel on top of the booster.

Preferably, the booster does not contain ammonium compounds.

A further object of the present disclosure is a multi-component unit (kit-of-parts) for the brightening/bleaching of keratinous fibers, which contains at least two separately packaged components, the first component (A) a peroxide hydrogel composition as described herein, and the second component (B) is a booster as described herein. Components (A) and (B) are preferably in a weight-related ratio (A):(B) of from about 1:0.8 to 1:2.5, more preferably 1:1 to 1:2.

As contemplated herein, the blonding powder of the 2-component kit is preferably composed in such a way that the mixture (i.e., the ready-to-use brightening mixture), has an alkaline pH value, preferably a pH value of from about 8.5 to about 12.0, particularly preferably a pH value of from about 9.0 to about 11.5, extremely preferably a pH value of from about 9.5 to about 11.0, in each case measured at about 25° C.

A multi-component unit comprises several individual components, which are assembled separately from each other, and a common packaging for these components, for example a folding carton. Here the components are provided separately in different containers. In the context of the present disclosure, a container is understood to be an enclosure in the form of a bottle, a tube, a can, a sachet, a sachet, or a similar wrapping, which may be re-closable. According to the present disclosure there are no limits to the wrapping material. Preferably, however, these are wrappings made of glass or plastic.

In addition, the packaging unit may include application aids such as combs, brushes or paint brushes, personal protective clothing, especially disposable gloves, and instructions for use. As mentioned, although it is preferable to mix the gel and booster in, e.g., a 1:1 to 1:2 ratio, alternatively, the booster may be applied to hair followed by subsequent application of the peroxide gel on top of the booster. In some embodiments, the hair is wrapped in foil, preferably aluminum foil, subsequent to the gel/booster application for the duration of the exposure time.

Preferably the exposure time is from about 5 to about 60 min, especially from about 15 to about 45 min, which can occur at room temperature or at an elevated temperature. The term “room temperature” denotes the temperature in the room in which a person usually uses a hair dye, i.e., usually a bathroom or a hairdressing salon, in which the temperature is in the range of 10-29° C.

During the exposure time of the agents on the fiber it can be advantageous to support the lightening process by adding heat. An exposure phase at room temperature is also according to the present disclosure. In particular, the temperature during the exposure time is between about 20° C. and about 60° C., preferably at least 30° C., more preferably about 40° C. The hair can, for example, covered with a warming hood or a radiant heater.

At the end of the lightening process, all components found on the keratin fibers are rinsed from the hair with water or a surfactant-based detergent. In particular, commercially available shampoo can be used as a cleaning agent, whereby the cleaning agent can be dispensed with and the rinsing process, or the rinsing process can be carried out with tap water if the lightening agent has a higher surfactant content.

The ability to lighten hair was determined in part by assessing the ΔE (DE) and ΔL (DL) values, where DE reflects the total color difference and DL measures the lightness difference. DE values were considered significant if higher than 1, and a lighter color is represented by a positive DL value, whereas a darker color is reflected by a negative DL value. The peroxide gels described herein provide color change and lift that is noticeable by eye, i.e., provide DE and DL values greater than 2. The gel+booster systems described herein are comparable and able to achieve a DE and DL that is within 5 units, more preferably 2 units, of hair treated with a non-gel peroxide+booster system.

The level of hair damage caused by bleaching systems can be measured in various ways including assessment of surface damage via SEM imaging, levels of cysteic acid and peak capacitance value. Cysteic acid levels were measured using a salon lab analyzer containing a NIR spectrophotometer sensor as disclosed in US20210068744.

The morphological changes of hair at the microstructural level can be assessed by using of scanning electron microscopy (SEM), transmission electron microscopy (TEM), and atomic force microscopy (AFM).

Use of the peroxide hydrogels described herein to lighten or to bleach hair in combination with a booster, results in less visible damage to hair fibers than is produced by traditional developers or bleaching systems in which hydrogen peroxide is not encapsulated in a hydrogel.

Cysteic acid residues, the oxidation product of cystine or disulfide bonds, is as an indicator of hair damage resulted from bleaching and oxidative coloring processes. When hair is exposed to chemical processes such as oxidative coloring or bleaching, changes occur firstly in the surface layers and in the removal of the 18-MEA. The reaction with 18-MEA results in the formation of acidic sulfur compounds, such as cysteic acid residues. Hair fiber properties, especially the hair surface, change from a hydrophobic to more hydrophilic characteristics. Bleach also oxidizes cystine residues of the hair matrix in the cortex and other hair regions rich in cystine such as the A-Layer and the exocuticle inside cuticle cells. These reactions result in the formation of cysteic acid residues and in the breakdown of the CMC, the cuticle and cortex components and ultimately dissolving proteins in these regions.

Use of the peroxide hydrogels described herein to lighten hair, or to bleach hair in combination with a booster, results in lower amounts of cysteic acid in treated hair as compared to hair lightened or bleached with hydrogen peroxide that is not contained in a hydrogel.

Peak capacitance provides information on the level of damage as well with a higher level of peak capacitance indicating more hair damage.

The peroxide hydrogels and systems show lower peak capacitance levels than traditional products in which hydrogen peroxide is not encapsulated in a hydrogel.

### Examples

### Example 1: Preparation Silica Gel-H₂O₂ Compositions

TABLE 1 shows exemplary compositions for preparing peroxide hydrogels for keratinous fibers.

TABLE 1

| Raw Materials | Range of Variation | |
|---|---|---|
| Calcium Chloride | 0-1.5 | wt. % |
| Sodium Silicate | 4-8 | g |
| Amberlite IR 120 (Hydrogen | 1.8-3.6 | g |
| form) ion exchange resins | | |
| Orthophosphoric Acid | 0-400 | uL |

* Orthophosphoric Acid is to
be added in two separate
steps. If too much added at
the beginning, it is difficult to
separate out the resins from
the final gel

A silica gel H₂O₂ composition suitable for keratin fibers and comprising sodium silicate, calcium chloride, hydrogen peroxide, heavy water (2H2O), phosphoric acid and demineralized water was prepared with the ion-exchange resin using the sol-gel method.

In one example, 32 g 1% Calcium Chloride (aq.) 8 g of Sodium Silicate was added. After mixing, 50 μL of Orthophosphoric Acid was added followed by Hydrogen peroxide in a manner that final concentration is 5 vol %. Then 1.8 g of Amberlite IR 120 (Hydrogen form) ion exchange resins was added to solution. The solution was stirred with magnetic stirrer for 4 min. The resins were then removed from the gel and 350 μL of orthophosphoric was added and the final gel was formed at a pH of 4. The resulting hydrogel contained Sodium Silicate 40/42 (17%), Calcium Chloride.2H₂O (0.68%), 50% H₂O₂ (12.78%), 85% Phosphoric Acid (1.6%), Water Demineralized (68%) and had good gel stability.

Gels prepared with less than 0.5% calcium chloride were more transparent, but the stability was not as good, while gels having closer to 1.5% of the calcium chloride were more sol like.

### Example 2: Application of Peroxide Gel to Hair

Strands of hair (Kerling, Euronaturhaar 6-0) were colorimetrically measured (Datacolor Spectraflash SF 450), pre-shampooed and dried.

The peroxide hydrogels of Example 1 were applied to 6.0 brown virgin hair swatches alone and in combination with a commercially available “blond booster.” The treated hair was wrapped in aluminum foil and developed for 15 minutes several days. The bleaching powder contained the following ingredients: Sodium Silicate, Magnesium Carbonate Hydroxide, Sodium hexametaphosphate, Hydroxyethylcellulose, Cellulose Gum, Tetrasodium EDTA, Silica, Ariabel Blue, Potassium persulfate, Ammonium persulfate+0.5% Silica, Succinic Acid, Lysine HCl, L-Arginine, Parfum Touch 815, Paraffinum Liquidum.

The gel and powder were mixed at a 1:1 or a 1:2 ratio before applying to the hair swatches. The treated swatches were wrapped in in aluminum foil and developed for 15 to minutes several days on a 40° C. temperature stage. Results are reported for 15, 30, and 60 minutes. The hair strands were rinsed out with water and then colorimetrically measured again.

Lightening Effectiveness: The ability to lighten hair was determined in part by assessing the ΔE (DE) and ΔL (DL) values, where DE reflects the total color difference and DL measures the lightness difference. DE values were considered significant if higher than 1, and a lighter color is represented by a positive DL value, whereas a darker color is reflected by a negative DL value.

Cysteic Acid content was evaluated using a salon lab analyzer containing a NIR spectrophotometer sensor as disclosed in US20210068744.

Results for the 17% hydrogen peroxide gel (without booster) are shown in TABLE 2. It provided visible lightening and lift at 15-, 30-, and 60-minutes exposure times, with the level of lift and lightening increasing with time. At all times, cysteic acid values indicate only slight damage.

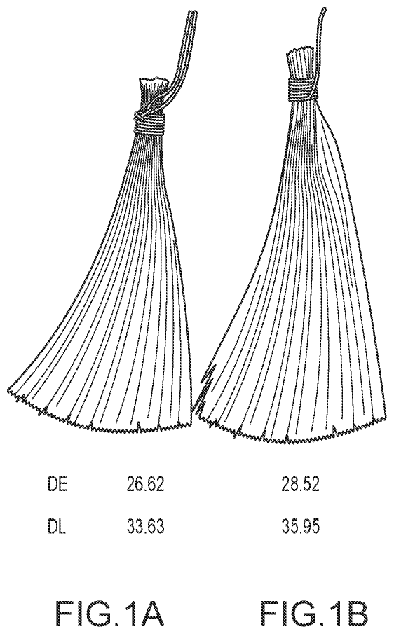

The effect of adding the booster is shown in FIG. 1, where a swatch after treatment with gel+lightening powder at 1:1 ratio was applied, and hair was developed for 30 minutes. The DE was 26.62 and DL was 33.63 indicating that the gel+booster provided a significant lightening/bleaching effect of the system.

### Example 3: Comparison of Hair Lightening and Associated Damage Using a Traditional Developer Versus Peroxide Hydrogel

Lightening and hair damage resulting from traditional versus hydrogel peroxide compositions were compared. The comparator contained 12.2% of H₂O₂ (49.5% stock). The virgin hair strands were the same as in Example 1. The compositions were left on hair for 30 minutes and then rinsed with water and colorimetrically measured.

The comparative “traditional” bleaching developer contained:Water, demineralized, Sodium hydroxide, Dipicolinic acid, Disodium pyrophosphate, Etidronic Acid, Xanthan Gum, Propanediol-1,2, Hydrogen peroxide.

The ability to lighten hair was determined in part by assessing the ΔE (DE) and ΔL (DL) values. The higher the DE value, the greater the color shift compared to untreated hair. The higher the DL value, the greater the lightening of the strands compared to untreated hair.

Treated hair swatches were viewed using a SEM microscope, and cysteic Acid content was evaluated using a Near-Infrared (NIR) Spectrometer with sensor. The spectra were recorded with [an MPA™ FT-NIR spectrometer from Bruker Optik GmbH. The infrared range covers the wave number range from 12500 cnr 1 to 4000 cnr 1 and is characteristic for overtone and combination vibrations of, for example, CH, OH and NH groups.

The wavenumber range from 7300 cm 1 to 4020 cm 1 was selected for the analysis of the measured NIR spectra. The NIR spectra of cystine show characteristic absorption bands in the wavenumber range from 6200 cm¹ to 5500 cm¹. If the hair changes due to greater damage (i.e., if the cysteic acid content in the hair increases), this affects the bands characteristic of cysteic acid at 5020 cm¹ to 4020 cm¹ in the NIR spectrum. The quantitative evaluation of the NIR spectra was computer-aided.]

### Results

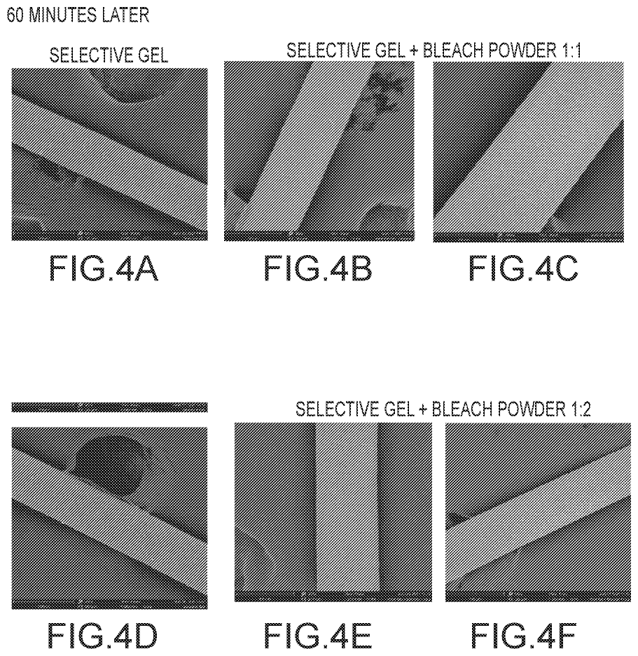

The hair was visibly lightened by the gel composition as confirmed by the DE and DL values in TABLE 2. FIGS. 4A and 4D show that after applying of the gel composition for 60 minutes, the hair has little to no visible damage. The cysteic acid levels shown in TABLE 2 are consistent with this visual observation.

TABLE 2

| | 17% Peroxide Hydrogel | | | Traditional Developer | | |
|---|---|---|---|---|---|---|
| | Application Time (min) | | | Application Time (min) | | |
| Test Result | 15 | 30 | 60 | 15 | 30 | 60 |
| DE | 3.52 | 3.65 | 6.50 | 1.14 | 1.75 | 3.73 |
| DL | 2.59 | 2.67 | 4.41 | 1.09 | 1.68 | 3.39 |
| Cysteic Acid | 1.21 | 1.44 | 1.60 | 1.63 | 2.28 | 2.23 |

Cysteic acid levels for the traditional developer indicated slight damage at 15 minutes and damaged hair at 30 and 60 minutes. The peroxide hydrogel provided greater lightening and lift at each timepoint compared to the traditional developer with less resulting damage.

### Example 4: Effect of a Booster Amount on Hydrogel Hair Bleaching Systems and Comparison with Traditional Bleaching System

The DE/DL values and resulting hair damage were also assessed and compared traditional bleaching system when the amount of booster was varied in the gel system. The gel was the 17% hydrogen peroxide gel of Example 1. The developer was the same as Example 2.

The booster bleaching powder contained:Sodium Silicate, Magnesium Carbonate Hydroxide, Sodium hexametaphosphate, Hydroxyethylcellulose, Cellulose Gum, Tetrasodium EDTA, Silica, Ariabel Blue, Potassium persulfate, Ammonium persulfate+0.5% Silica, Succinic Acid, Lysine HCl, L-Arginine, Parfum Touch 815, Paraffinum Liquidum.

The developer+powder was combined at the recommended 1:2 ratio and gel+powder were combined at a 1:1 or a 1:2 ratio before applying to the hair swatches. The treated swatches were wrapped in in aluminum foil and exposed for 15 minutes to 60 minutes on a 40° C. temperature stage.

As shown in FIGS. 1A and 1B, the hydrogel peroxide composition lightens hair just as effectively as the traditional developer, indicated by the similar DE and DL values. Specifically, the virgin hair treated with application of the hydrogel+booster system of Example 1 had a DE value of 26.62 and a DL value of 33.63 (see FIG. 1A), whereas virgin hair dyed with the traditional bleaching system had a DE value of 28.52 and a DL value of 35.95 (see FIG. 1B).

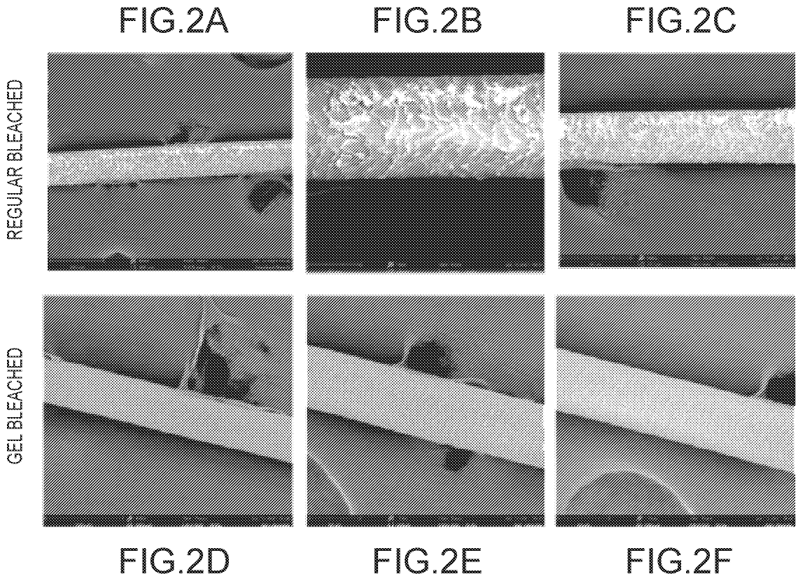

The hair treated by application of gel+booster system in a 1:1 ratio for 60 minutes also experienced less overall hair damage as seen in FIGS. 2D through 2F. Specifically, comparing SEM images of gel bleached hair and traditional bleached hair show that gel bleached hair exhibited less surface damage (see FIGS. 2D through 2F) than hair bleached using a traditional bleaching system (see FIGS. 2A through 2C).

Similarly, as shown in TABLE 3 the hair bleached with the traditional bleaching system experienced more damage compared to the hair bleached using the hydrogel bleaching system indicated by the higher cysteic acid and peak capacitance levels.

TABLE 3

| | Cysteic Acid | Peak Capacitance |
|---|---|---|
| Sample | (wt. %) | (Fx10⁻⁸) |
| Virgin Hair | 1.22 | 0.39 |
| Hydrogel + Booster | 2.97 | 0.86 |
| Developer + Booster | 4.76 | 13.86 |

Notably, the hair bleached using the hydrogel system at a 1:2 ratio for 60 minutes provides the same lightening effect as the traditional bleaching system (evidenced by the similar DE and DL values) with less overall hair damage as evidenced by the SEM imaging in FIGS. 2A through 2F and cystic acid/peak capacitance values in TABLE 3. Specifically, not only is there less visible damage on the surface of hair via SEM, but there are also lower levels of cysteic acid and a lower peak capacitance on the gel bleached hair.

Moreover, this trend continued even when ratio of peroxide hydrogel to booster was varied. As shown in TABLE 4, the hydrogel bleaching system achieved greater lightening effect when used in conjunction with the booster. When the gel/booster was used in a 1:1 ratio, DE=47.57 and DL=43.26. When the gel/booster was used in a 1:2 ratio, DE=49.95 and DL=45.07. Both gel/booster ratios achieved greater lightening than the traditional bleach/booster system.

TABLE 4

| | Time | | | | | |
|---|---|---|---|---|---|---|
| | 15 min | | 30 min | | 60 min | |
| | DE | DL | DE | DL | DE | DL |
| Traditional Developer | 1.14 | 1.09 | 1.75 | 1.68 | 3.73 | 3.39 |
| Peroxide Hydrogel | 3.52 | 2.59 | 3.65 | 2.67 | 6.50 | 4.41 |
| Developer + Booster (1:2) | 24.10 | 18.78 | 40.20 | 34.54 | 42.97 | 37.64 |
| Gel + Booster (1:1) | 27.27 | 21.87 | 31.01 | 26.11 | 47.57 | 43.26 |
| Gel + Booster (1:2) | 26.53 | 20.61 | 36.96 | 30.31 | 49.95 | 45.07 |

TABLE 5

| | Cysteic Acid (wt. %) | | |
|---|---|---|---|
| | 15 min | 30 min | 60 min |
| Traditional Developer | 1.63 | 2.28 | 2.23 |
| Gel Only | 1.21 | 1.44 | 1.60 |
| Developer + Booster (1:2) | 5.86 | 5.11 | 6.29 |
| Gel + Booster (1:1) | 1.83 | 2.25 | 3.50 |
| Gel + Booster (1:2) | 1.90 | 2.69 | 3.63 |

TABLES 4 and 5 show that the hydrogel bleaching system is able to achieve a higher DE and DL (i.e., greater lightening effect) while also being less damaging on hair, reflected by lower cysteic acid levels (TABLE 5).

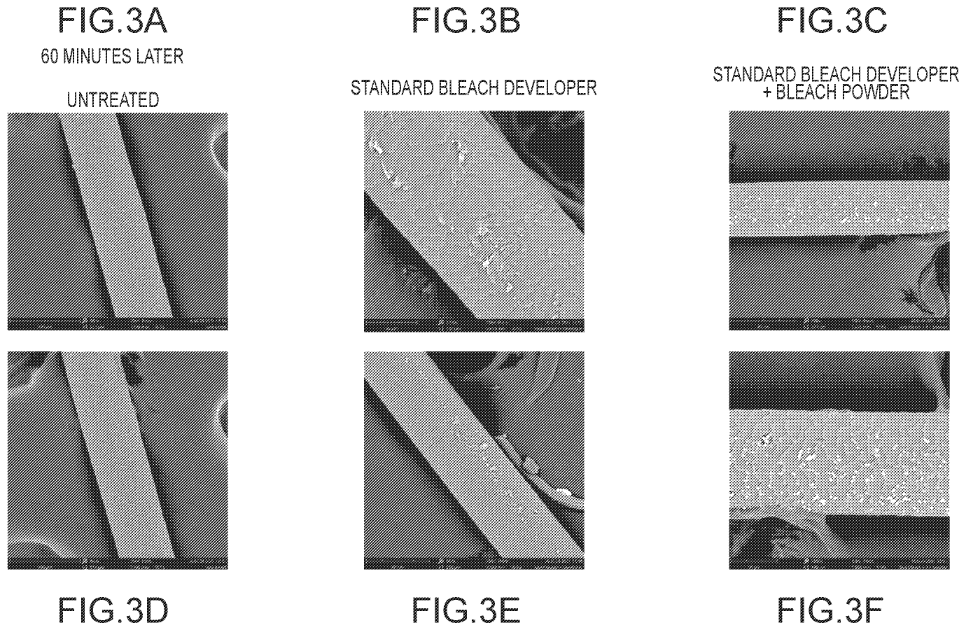

In addition to the cysteic acid evaluation, the gel bleached hair was also found to have less surface damage via SEM imaging as shown in FIG. 4. Comparing FIGS. 3B/C and 3E/F with FIGS. 4B/C and 4E/F, it is evident that hair bleached using the gel bleaching system is far less damaging to the hair's surface.

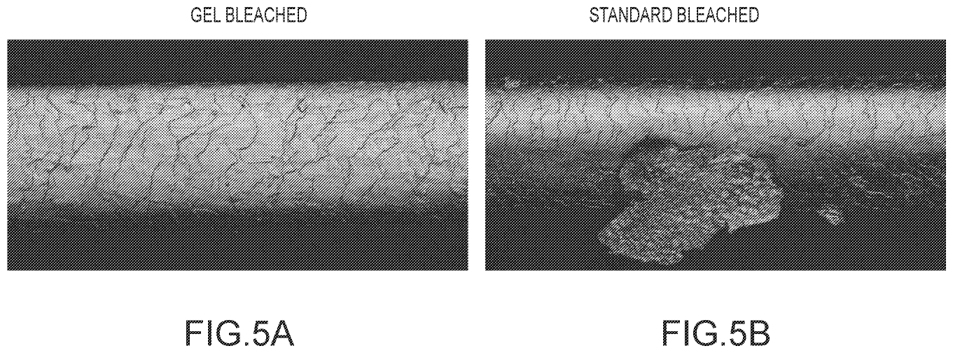

Furthermore, as shown in FIGS. 5A and 5B, the hair bleaching using a traditional bleaching system with booster (FIG. 5B) was found to have broken cuticles when viewed microscopically, whereas the hair bleached using a peroxide hydrogel bleaching composition (FIG. 5A) did not show cuticle damage.

Overall, all evidence indicates that a hydrogel bleaching system is more effective at bleaching hair, indicated by a higher DE and DL, while simultaneously resulting in less overall hair damage indicated by lower cysteic acid levels and less surface damage via SEM imaging. Without wishing to be bound by this theory, it is believed that the hydrogel system results in less hair damage since unlike traditional bleaching systems, which directly attack the hair's melanin, the hydrogel in the hydrogel bleaching system encapsulates the bleach and does not directly attack the hair's melanin resulting in a decrease in the negative effects associated with traditional bleaching systems, i.e., poor hair feel and look, reduced hair strength, increased incidents of split ends and breakage.

1. A system for lightening keratinous fibers, the system comprising:
a peroxide hydrogel composition; and
an oxidizing agent booster,
wherein, when the peroxide hydrogel composition and the oxidizing agent booster are combined in a ratio of from 1:1 to 1:2, the system has a pH of from about 9 to about 12.

2. The system of claim 1, wherein the peroxide hydrogel composition comprises hydrogen peroxide in an amount of from about 5 to about 17% by weight, based on the total weight of the peroxide hydrogel composition.

3. The system of claim 1, wherein the peroxide hydrogel composition comprises sodium silicate.

4. The system of claim 3, wherein the sodium silicate is in an amount of from about 15 to about 30% by weight, based on the total weight of the peroxide hydrogel composition.

5. The system of claim 1, wherein the peroxide hydrogel composition comprises a metal cation.

6. The system of claim 5, wherein the metal cation is divalent.

7. (canceled)

8. The system of claim 1, wherein the peroxide hydrogel composition comprises at least one of calcium chloride in an amount of from about 0.1 to about 1% by weight or phosphoric acid in an amount of from about 0.5 to about 2.5% by weight, based on the total weight of the peroxide hydrogel composition.

9. (canceled)

10. The system of claim 1, wherein the peroxide hydrogel composition has a pH of from about 3.5 to about 4.5 prior to being combined with the oxidizing agent booster.

11. The system of claim 1, wherein the oxidizing agent booster comprises a persulfate selected from inorganic salts of a peroxosulfuric acid and mixtures thereof, the persulfate being in an amount of from about 5 to about 85% by weight, based on the total weight of the oxidizing agent booster.

12-13. (canceled)

14. A kit for bleaching hair, the kit comprising:
an oxidizing agent booster having a pH of from about 9 to about 12; and
a peroxide hydrogel having a pH of from about 3 to about 4.5,
where the oxidizing agent booster and the peroxide hydrogel are in a weight ratio of 1:1 to about 2:1, based on the total weight of the kit.

15. The kit of claim 14, further comprising at least one of an application aid, disposable gloves, or instructions for use for the kit.

16-21. (canceled)

22. A cosmetic peroxide hydrogel composition comprising:
hydrogen peroxide in an amount of from about 5 to about 17% by weight, based on the total weight of the cosmetic peroxide hydrogel composition;
a metal silicate; and
a metal cation or a salt thereof,
wherein the cosmetic peroxide hydrogel composition has a pH of from about 3 to about 4.5.

23. The cosmetic peroxide hydrogel composition of claim 22, wherein the metal silicate is sodium silicate.

24. The cosmetic peroxide hydrogel composition of claim 23, wherein the sodium silicate is in an amount of from about 15 to about 30% by weight, based on the total weight of the cosmetic peroxide hydrogel composition.

25. The cosmetic peroxide hydrogel composition of claim 23, wherein the metal cation or the salt thereof is a divalent metal cation salt.

26. The cosmetic peroxide hydrogel composition of claim 25, wherein the divalent metal cation salt is a calcium cation salt.

27. The cosmetic peroxide hydrogel composition of claim 26, wherein the calcium cation salt is calcium chloride, the calcium chloride being in an amount of from about 0.1 to about 1% by weight, based on the total weight of the cosmetic peroxide hydrogel composition.

28. The cosmetic peroxide hydrogel composition of claim 22, further comprising phosphoric acid in an amount of from about 0.5 to about 2.5% by weight, based on the total weight of the cosmetic peroxide hydrogel composition.

29. A method of lighting a keratinous fiber, the method comprising:
applying the cosmetic peroxide hydrogel composition of claim 22 to the keratinous fiber for a time of from 5 to 60 minutes.

30. A method of bleaching a keratinous fiber, the method comprising:
mixing the cosmetic peroxide hydrogel composition of claim 22 with an oxidizing agent booster to form a ready-to-use mixture; and
applying the ready-to-use mixture to the keratinous fiber for a time of from 5 to 60 minutes.

31-35. (canceled)
